(19) United States
(12) Patent Application Publication
(10) Pub. No.: US 2022/0211311 A1
(43) Pub. Date: Jul. 7, 2022

(54) SYSTEM AND METHOD FOR FACILITATING COMMUNICATION BETWEEN PARTIES

(71) Applicant: Kathryn M. Laurance, Houston, TX (US)

(72) Inventor: Kathryn M. Laurance, Houston, TX (US)

(21) Appl. No.: 17/142,255

(22) Filed: Jan. 6, 2021

(51) Int. Cl. A61B 5/16; A61B 5/246

(52) U.S. Cl. CPC A61B 5/165; A61B 5/246

(57) ABSTRACT

System and method to assist two parties to communicate effectively by using an intermediary to project their emotions through a combination of telepathy and empathy, thus synchronizing the adverse parties' emotional states whereby this particular intermediary may be a person capable of natural empathic telepathy.

### TECHNICAL FIELD

The system and method of the present invention relate to the fields of communications; more particularly, the present invention relates to using an intermediary for improving communication between adversative parties.

### BACKGROUND

Communication is the process of reaching a mutual understanding between two or more parties, where participants not only exchange information but also create and share meaning between each other. Communication is about understanding one another's emotion and intentions behind the information. Learning how to properly communicate is an important ability in life that enables people to better understand and relate to those around them. There are many occasions in our everyday life that requires communication and reading another person's emotion. Communication can separate a bad date from a good one, help get your dream job, and reach out to your kids. Communication is also important on a global scale that can establish nations, finish peace settlements, make treaties, and define history.

The better the parties are at communication the better the outcome will be. The problem most parties experience when trying to utilize effective communication come from inadequacies with their emotional intelligence. Most disagreements are based on emotion as opposed to reason, which can often take an enormous amount of time and money to subside. People have trouble reading emotions accurately, evaluating and assembling different emotions in a useful way, predicting how present and future events will affect their emotions, and regulating their emotions when they experience a strong disturbance. These inadequacies can lead to a larger divide between the parties and prevent a resolution from ultimately occurring.

### SUMMARY

The present invention solves the problem with parties not being able to communicate effectively by using an intermediary to project their emotions through a combination of telepathy and empathy, thus synchronizing the adverse parties' emotional states. This particular intermediary may be a person capable of natural empathic telepathy.

There are different methods people have been using to explore empathic telepathy, such as artificial telepathy, where a computer can pick up electromagnetic signals from the brain containing thoughts and emotions and translate this information into binary code to be displayed or transferred. There also has been research with humans trying to communicate with dolphins thinking dolphins can communicate by empathic telepathy because of their excess brain matter and advanced communication skills. Some people are naturally capable of empathic telepathy and can project or receive thoughts, memories, emotions, and the perception of human senses including taste, smell, touch, sight, and hearing, voluntarily or sometimes involuntarily. How intense these forms of communication from the person capable of empathic telepathy (PCET) are projected onto another person or party is based on how strong the emotion the PCET is feeling, how vividly the PCET remembers a memory, how much a PCET is thinking about a particular thought, or how intense a sensation is perceived.

Display of these abilities can be a double-edged sword for the PCET emitting them because confidential information the PCET is hiding may be disclosed without their permission but also may help a party reach a desired emotional state they could not obtain on their own. For instance, if a party is feeling anxiety, the PCET can physically ask or telepathically converse with the party on why they are feeling that way, then can work out the parties' emotional needs, and transmit the needed emotion to the party. This can also be beneficial when a PCET is trying to harmonize adverse parties who can't come to an agreement because of their emotional states. If both parties are in a state of anger because of frustration at the lack of progress in negotiations the PCET can project an aura of calm and tranquility that will supersede the parties' anger and help facilitate communication.

### BRIEF DESCRIPTION OF DRAWINGS

FIG. 1 illustrates a PCET intermediary synchronizing the parties' emotional states with each other.

### DETAILED DESCRIPTION

In the Summary above and in this Detailed Description, and the claims below, and in the accompanying drawings, reference is made to particular features of the invention. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range including that number (which may be a range having an upper limit or no upper limit, depending on the variable being defined). For example, “at least 1” means 1 or more than 1. The term “at most” followed by a number is used herein to denote the end of a range, including that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limits include both numbers. For example, “25 to 100” means a range whose lower limit is 25 and upper limit is 100, and includes both 25 and 100.

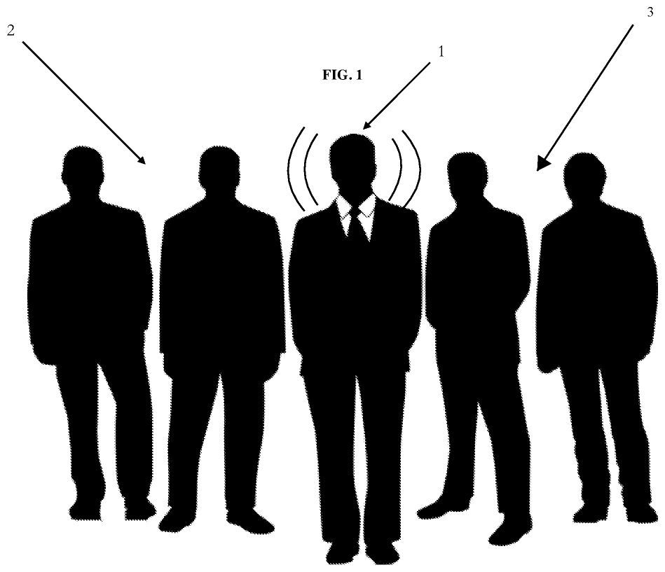

The present invention is a system and method of using a person capable of empathic telepathy (PCET) as an intermediary to help facilitate conversation between two or more parties who are having problems coming to an agreement or cannot come to a common goal. Referring now to the drawing figures, there is shown in FIG. 1 a system where the intermediary is a PCET 1 and projects their thoughts, emotions, feelings, or senses onto two or more parties 2 and 3 to synchronize the parties' 2 and 3 emotional states. To achieve this the PCET 1 can think of stimuli such as specific memory, recall an event that occurred, perceive or remember a specific type of sense such a smell, or watch or listen to something that can help the PCET feel the desired emotion enough to transfer the emotion to the parties 2 and 3. The range and intensity can be increased or decreased based on how strong the emotion is or how strong the PCET's reaction to the stimuli is. The PCET can halt the projection voluntarily after the desired results have been reached. As shown in FIG. 1 a method for facilitating communication between parties using the system as described above is also disclosed. The method includes the steps of providing the system where the intermediary is a person who is capable of empathic telepathy 1 and projects their thoughts, emotions, feelings, or senses, onto two or more parties 2 and 3 to synchronize the parties' 2 and 3 emotional states.

1. A system for facilitating communication comprising:
an intermediary who is a person capable of empathic telepathy; and
two or more parties to receive communication from said intermediary, wherein the intermediary facilitates conversation between two or more parties who are having problems coming to an agreement or cannot come to a common goal.

2. The system of claim 1 wherein the intermediary uses a stimuli to provide the desired emotion enough to transfer the emotion to the parties.

3. The system of claim 2 wherein the stimuli is a specific memory.

4. The system of claim 2 wherein the stimuli is an event recalled.

5. The system of claim 2 wherein the stimuli is a specific type of sense such a smell.

6. The system of claim 2 wherein the range and intensity of the empathetic telepathy is increased or decreased based on how strong an emotion is or how strong the intermediary's reaction to the stimuli.

7. The system of claim 2 wherein the stimuli is achieved by watching or listening to something that can help the intermediary feel.

8. The system of claim 7 wherein the intermediary halts the empathetic telepathy voluntarily after the desired results have been reached.

9. A method for facilitating communication comprising:
obtaining an intermediary who is a person capable of empathic telepathy; and
obtaining two or more parties to receive communication from said intermediary, wherein the intermediary facilitates conversation between two or more parties who are having problems coming to an agreement or cannot come to a common goal.

10. The method of claim 8 further comprising using a stimuli to provide the desired emotion enough to transfer the emotion to the parties.

11. The method of claim 9 wherein the stimuli is recalling a specific memory.

12. The method of claim 9 wherein the stimuli is recalling a specific event.

13. The method of claim 9 wherein the stimuli is smelling an object.

14. The method of claim 9 wherein the stimuli is watching or listening to something that can help the intermediary feel.

15. The method of claim 9 adjusting range and intensity of empathetic telepathy based on how strong an emotion is or how strong the intermediary's reaction to the stimuli.

16. The method of claim 7 further comprising halt the empathetic telepathy voluntarily after the desired results have been reached.
